(19) United States
(12) Patent Application Publication
(10) Pub. No.: US 2016/0328950 A1
(43) Pub. Date: Nov. 10, 2016

(54) METHOD FOR MANAGING A POOL OF EMERGENCY DEVICES, ASSOCIATED EMERGENCY DEVICE AND SERVER FOR MANAGING A POOL OF EMERGENCY DEVICES

(71) Applicant: BULL SAS, Les Clayes Sous Bois (FR)

(72) Inventors: Benoît PELLETIER, Saint Etienne de Crossey (FR); Landry Stéphane ZENG EYINDANGA, Grenoble (FR); Christian BOURGEOIS, Saint Etienne de Crossey (FR)

(21) Appl. No.: 14/947,712

(22) Filed: Nov. 20, 2015

(30) Foreign Application Priority Data
Nov. 20, 2014 (FR) 14 61277

(51) Int. Cl. G08B 25/01; A61N 1/39; G08B 7/06

(52) U.S. Cl. CPC G08B 25/016; G08B 7/066; A61N 1/39

(57) ABSTRACT

A method for managing a pool of emergency devices, comprising: determining the location of a monitoring device carried by a victim experiencing a medical problem, locating, in the pool of emergency devices, at least one emergency device that is near the located monitoring device, an alarm step that indicates at least the position of the located emergency device, a guiding step that indicates, at the located emergency device, at least the position of the located monitoring device.

### RELATED APPLICATION

The present application claims priority to French Application No. 14 61277 filed Nov. 20, 2014, which is incorporated herein in its entirety by reference.

### FIELD OF THE INVENTION

The invention concerns a method for managing a pool of emergency devices, a server for managing a pool of emergency devices, and an emergency device. The emergency device is advantageously a cardiac defibrillator.

### BACKGROUND OF THE INVENTION

In the prior art, in which the emergency devices are cardiac defibrillators, a system is known wherein cities and/or public agencies and/or companies deploy self-service cardiac defibrillators within their respective districts, facilities, and premises.

When someone then experiences a heart attack, witnesses must identify who among them has medical training or must quickly find someone nearby who does. Once the rescuer has been identified, he or she begins looking for a defibrillator in the vicinity so that action can be taken as quickly as possible. The rescuer is not necessarily a physician. He or she may only have basic first aid training

In addition, the rescuer will not necessarily be someone who knows the geographical area where the heart attack has occurred. In particular, the rescuer will not systematically have access to a map of available defibrillators close to the site of the heart attack. The rescuer can therefore lose precious time searching for the nearest defibrillator or for another defibrillator that is also relatively close.

Response time is critical for the heart attack victim, however. If defibrillation is performed quickly, preferably within the first few minutes after fibrillation begins, the chances of survival are significantly increased.

A map of cardiac defibrillators is often accessible on the Internet (for example on a site that publishes freely accessible data referred to as “Open Data”). The rescuer can then use this map to find the emergency device closest to the incident, but will lose time conducting this explicit search, a loss of time that could be fatal to the victim.

### SUMMARY OF THE INVENTION

The aim of the present invention is to provide a method for managing a pool of emergency devices which at least partially overcomes the above disadvantages.

More particularly, the invention aims to provide a method for managing a pool of emergency devices which tends to reduce the time until rescuers can begin assisting victims of a health emergency requiring the use of an emergency device not in the possession of the rescuers.

More particularly, the invention aims to provide a method for managing a pool of emergency devices, which will better distribute and share information between the various elements of the network, namely the server, the monitoring device, and the emergency devices, and possibly also telecommunications devices, to allow decreasing the total travel time before medical aid is rendered, meaning the time necessary to bring at least one emergency device and at least one rescuer to the location of the victim's monitoring device.

More particularly, the invention aims to provide a method for managing a pool of emergency devices which will first concentrate on the emergency device and its proximity to the monitoring device from which the signal used to locate the victim originated, rather than on the identified rescuers and their proximity to the monitoring device from which the signal used to locate the victim originated, in order to reduce the travel time before aid is rendered and to facilitate the intervention of rescuers even if they are not registered.

The fact that the first focus is on the emergency device close to the monitoring device from which the signal used to locate a victim originated, rather than on the available rescuer or rescuers in the vicinity, as well as on optimization of the route between the emergency device and the monitoring device from which the signal used to locate a victim originated, which is actually more critical than the route between rescuer and monitoring device from which the signal used to locate a victim originated, allows reducing the actual travel time before aid is rendered and giving priority to contacting one or more rescuers who are near the emergency device rather than near the monitoring device from which the signal used to locate a victim originated.

To this end, the invention provides a method for managing a pool of emergency devices, comprising: a step of determining the location of a monitoring device carried by a person experiencing a medical problem, a step of locating, in the pool of emergency devices, at least one emergency device near the located monitoring device, an alarm step that indicates at least the position of the located emergency device, a guiding step that indicates, at the located emergency device, at least the position of the located monitoring device.

To this end, the invention also provides a server for managing a pool of emergency devices, suitable for carrying out: a step of determining the location of a monitoring device carried by a person experiencing a medical problem, a step of locating, in the pool of emergency devices, an emergency device near the located monitoring device, an alarm step that indicates at least the position of the located emergency device, a guiding step that indicates, at the located emergency device, at least the position of the located monitoring device.

To this end, the invention further provides an emergency device intended to be placed in a pool of emergency devices, and adapted for carrying out, on a server for managing the pool of emergency devices, when said emergency device has been located as being close to a monitoring device carried by a victim experiencing a medical problem: an alarm step, triggered by a command from the management server, that indicates at least the position of said located emergency device, a guiding step that indicates at least the position of the monitoring device after receiving this position from the management server.

In preferred embodiments, the invention comprises one or more of the following features, used separately or some or all of them in combination, in association with any of the objects of the invention described above.

Preferably, the method for managing a pool of emergency devices also includes a step of locating at least one telecommunications device carried by a rescuer who is near the located emergency device, and a step of sending, to the located telecommunications device, at least the position of the located emergency device. This gives priority to contacting a rescuer who is near the emergency device and who is therefore able to lay hands on this emergency device more quickly and then head more directly to the monitoring device from which the signal used to locate a victim originated, rather than a rescuer near the victim but who does not know how and where to obtain an emergency device.

Preferably, in the sending step, a route to the located emergency device is sent to the located telecommunications device, this route preferably being recalculated in real time according to any movement of the located emergency device. Thus, the notified rescuer can be brought as quickly as possible to the emergency device closest to the victim, while shortening the travel time if the emergency device is already being transported to the victim.

Preferably, in the sending step, at least the position of the located monitoring device is sent to the located telecommunications device, preferably a route to the located monitoring device is sent to the located telecommunications device. Thus, the rescuer can have all the location information on his or her telecommunications device: both the information concerning the emergency device and that concerning the monitoring device from which the signal used to locate a victim originated.

Preferably, in the alarm step, a visual and/or audible alarm is/are triggered at the located emergency device, and in the guiding step at least a position of the located monitoring device is indicated at the located emergency device, preferably indicating a route to the located monitoring device. Thus, on the one hand all potential rescuers in the vicinity of the emergency device who are thus more likely to bring it as quickly as possible to the victim, even if they are not registered and even if they do not carry a telecommunications device, will be contacted immediately, and on the other hand once the emergency device is reached, this takes over to guide the rescuer or rescuers to the victim.

Preferably, said route or routes are the fastest in terms of travel time for the located emergency device and/or for the located telecommunications device, this travel time preferably being less than a predetermined duration, this predetermined duration even more preferably being less than 10 minutes or even less than 5 minutes. This advantageously optimizes the total travel time to allow at least one emergency device and at least one rescuer to meet at the site of the incident where the victim will be found, whether the emergency device is brought to the victim by the rescuer who will render aid or by someone else. Inside buildings or in urban areas, this travel time will be the travel time on foot, while in rural areas it may be the travel time by vehicle.

Preferably in the guiding step and/or in the sending step, other additional information may be indicated, including: the position or positions of the one or more located emergency devices, and/or the position or positions of the one or more other located telecommunications devices, and/or the medical qualification profile of the one or more other located telecommunications devices, and/or the identification of the monitoring device towards which the or each located emergency device is heading, and/or a confirmation of emergency response by one or more located telecommunications devices. All this additional information helps optimize the management of the incident, for example enabling an intelligent use of multiple rescuers having different profiles, for example both a simple first aid responder arriving first at the scene of the incident and a physician who is of course more qualified but who can only arrive later on the scene, or to further reduce the travel time before aid is rendered in the event that a non-medically trained person is closer to the incident than a rescuer.

Preferably, in the step of locating the telecommunications device, the telecommunications device or devices is or are the telecommunications device or devices closest to the located emergency device, the located telecommunications device or devices preferably being determined from among a predetermined group of telecommunications devices carried by rescue workers and registered. This allows further reducing the travel time before aid is rendered, and possibly warning as soon as possible the registered rescuers when necessary even if they are not in the immediate vicinity of the emergency device.

Preferably, the steps of the method are conducted in parallel with at least two located emergency devices and/or with at least two located telecommunications devices per located emergency device. This increases the chances of having at least one emergency device and at least one rescuer only a short time away from the victim, regardless of unexpected problems along their routes.

Preferably, movement of the located emergency device towards the located monitoring device and/or the arrival of the located emergency device at the located monitoring device, are indicated to the located telecommunications device and/or to other telecommunications devices carried by rescuers and alerted. The rescuer can thus, using their telecommunications device, change their route in real time based on the relative position of the victim who is no longer moving and the emergency device which is moving toward the victim, to further reduce travel time to the incident so as to arrive at the same time or almost at the same time as the emergency device being brought by a person without medical training.

Preferably, the step of locating a monitoring device is preceded by a step of identifying this monitoring device by analyzing data sent by the monitoring device indicating a medical problem in the person who is carrying said monitoring device. Preferably, the monitoring device sends data to a server which manages the pool of emergency devices within which this monitoring device is currently located, said server being able, through analysis of these data, to identify and locate the monitoring device. Thus, the managing server can initiate the warning process at soon as possible after the incident, but does so intelligently. “Intelligently” is understood to mean only if there is an actual incident or a true risk of an incident, and not at every turn, which in the long run would be detrimental to the effectiveness of the method as the various rescuers would become less responsive because of having been contacted too often for no reason. As an optional alternative, the step of locating a monitoring device is performed directly after transmission by the monitoring device of its geolocation, said monitoring device having itself performed a data analysis revealing a medical problem in the person who is carrying said monitoring device.

Preferably, two located telecommunications devices are placed in telecommunication with each other, the first located telecommunications device to reach the located monitoring device corresponding to a medical qualification profile that is lower than that of the second located telecommunications device which has not yet reached the located monitoring device. This allows, for example, intelligent use of multiple rescuers having different profiles, such as a rescuer knowing simple first aid reaching the scene of the incident first and a physician who is of course more qualified but only arrives at the scene of the incident at a later time. In this case, the physician en route to the incident can already be giving oral instructions to a first aid rescuer already at the scene concerning the most urgent steps to be taken first, and can be ready to take over as soon as he or she arrives at the scene.

Preferably, the emergency device is a cardiac defibrillator. As today's pools of defibrillators are expanding and continue to expand, fibrillation events are serious but can be treated by rescuers knowing simple first aid provided that the rescuers reach the site of the incident in time.

The emergency device can advantageously also be any other emergency device, besides a cardiac defibrillator, distributed within a pool of one or more emergency devices and which can be used to treat a serious incident by a rescuer knowing simple first aid or even a rescuer without training, meaning that the emergency device is very simple to use even for non-medical personnel but requires rapid intervention at the scene of the incident.

Other features and advantages of the invention will become apparent from reading the following description of a preferred embodiment of the invention, given by way of example and with reference to the accompanying drawings.

### BRIEF DESCRIPTION OF DRAWINGS

FIG. 1 schematically illustrates an exemplary system for carrying out the method for managing a pool of cardiac defibrillators according to one embodiment of the invention.

FIG. 2 schematically illustrates an exemplary execution of the method for managing a pool of cardiac defibrillators according to one embodiment of the invention.

### DETAILED DESCRIPTION OF THE INVENTION

In the following text, the emergency device will be considered to be a defibrillator, but the invention could also be applied to some other type of emergency device. Without reducing the generality of the method according to the invention, the telecommunications device is considered to be a conventional cell phone or smartphone, a smart watch, a tablet, a pager such as an on-call pager, or any other appropriate telecommunications device.

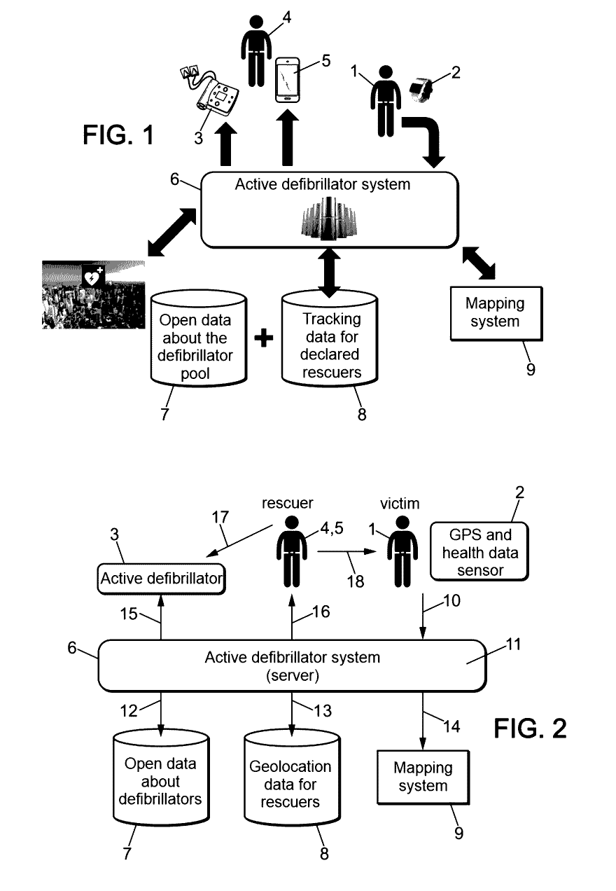

FIG. 1 schematically illustrates an exemplary system suitable for carrying out the method for managing a pool of cardiac defibrillators according to one embodiment of the invention.

A person potentially in need of aid (victim) 1 carries a monitoring device 2. One or more identified potential rescuers 4 each carry a cell phone 5. Other impromptu rescuers 4 may participate, but they must have been alerted by the alarm at the defibrillator 3. If a rescuer 4 is not carrying his or her cell phone 5 or has not turned it on, that rescuer will only participate as an impromptu rescuer alerted by the alarm at the defibrillator 3. The victim 1 and the rescuers 4 are in neighboring areas within a pool of defibrillators 3. The pool of defibrillators 3 is managed by a server 6 which has access to: a database 7 containing the distribution of the defibrillators 3 within the pool, a database 8 identifying a plurality of declared rescuers 4, a mapping system 9 to assist in locating in real time the monitoring device 2 of the victim 1 and the cell phones 5 of the rescuers 4 as well as the one or more defibrillators 3 moving toward the scene of the incident where the victim 1 is located.

The monitoring device 2 of the potential victim 1 of a heart attack comprises a health data sensor and a GPS (for “Global Positioning System”) geolocation sensor as well as a telecommunications module.

The health data sensor, which captures data including heart rate and blood pressure, is adapted to send these health data periodically to the server 6. Such a sensor is, for example, an “Apple Watch” (registered trademark). These health data can then be sent to the server 6 using a mobile application.

The geolocation sensor identifies the coordinates of the victim 1 and sends them to the server 6 via the telecommunications module, for example using a mobile application.

The cell phone 5 of the rescuer 4 has a mobile application to track the location of the rescuer 4 and notify him or her of the occurrence of a nearby incident.

The active defibrillator 3 comprises an alarm and a guiding display.

The alarm preferably includes both an audible alarm and a flashing light, both remotely controllable from the server 6.

The guiding display comprises a guiding system which guides toward the monitoring device 2 of the victim 1, and can be controlled remotely by the server 6. The server 6 sends, to the active defibrillator 3, the steps of the route to take from this active defibrillator 3 to the scene of the incident which is where the monitoring device 2 of the victim 1 is located. The active defibrillator 3 is the defibrillator 3 which was located by the server 6. This guiding system itself includes a GPS tracking system in order to adjust the route as the rescuer 4 travels toward the site of the incident, with calculation of the adjusted route constantly being performed by the server 6.

The server 6 is advantageously a real or virtual computer server that hosts the logic of the system for managing the pool of active defibrillators 3. Several functions are integrated within this logic. A first function is the detection of a serious cardiac anomaly from the health data received from the monitoring device 2 of a potential victim 1, or from each monitoring device 2 respectively belonging to different potential victims 1. A second function is the search for the active defibrillator 3 closest to the monitoring device 2 from which the signal used to locate the victim 1 originated. The third function is the remote triggering of the alarm of the active defibrillator 3 located as being closest to the site of the incident. The fourth function is the calculation of the fastest route between the located defibrillator 3 and the monitoring device 2 carried by the victim 1 at the site of the incident.

The server 6 also communicates with an open database 7 mapping the pool of installed defibrillators 3 (for example of the “Open Data” type (trademark)).

The server 6 also communicates with a database 8 of geolocation and contact information for the declared rescuers, who are therefore registered in it.

The server 6 also communicates with a mapping system 9 accessible from a programmable interface (for example such as “Google Maps” (trademark)).

FIG. 2 schematically illustrates an exemplary execution of a method for managing a pool of cardiac defibrillators according to one embodiment of the invention. The different “times” defined below either follow one another in generally rapid succession, or some may overlap with adjacent times, or may even be reversed. In particular, some of the steps which can be performed in parallel, meaning simultaneously, could be reversed.

In the same manner as in FIG. 1, a potential victim 1 carries a monitoring device 2. One or more identified potential rescuers 4 each carry a cell phone 5. The victim 1 and the rescuer or rescuers 4 are in neighboring areas within a pool of defibrillators 3. The pool of defibrillators 3 is managed by a server 6 which has access to: a database 7 containing the distribution of the defibrillators 3 within the pool, to a database 8 identifying a plurality of declared rescuers 4, a mapping system 9 to assist in locating in real time the monitoring device 2 of the victim 1 and the cell phones 5 of the rescuers 4 as well as the one or more defibrillators 3 moving toward the scene of the incident where the victim 1 is located.

In a first step 10, the monitoring device 2 collects and sends health data and geolocation data concerning the victim 1. These data respectively originate from the health data sensor and the geolocation sensor comprised in the monitoring device 2.

In a second step 11, the server 6 detects and identifies the victim 1 of a heart attack. To do so, the server 6 determines that the victim has suffered a heart attack or is in imminent risk of a heart attack, based on an analysis of the data from the health sensor being sent by the monitoring device 2. The server 6 also determines the location of the monitoring device 2, for example using geolocation coordinates provided by the monitoring device 2.

In a third step 12, the server 6 searches the database 8 containing the distribution of defibrillators within the pool, for the defibrillator 3 closest to the monitoring device 2 which is at the site of the incident suffered by the victim 1.

In a fourth step 13, the server 6 searches the 8 database identifying a plurality of declared rescuers 4, to find the rescuer or rescuers 4 closest to the victim 1 by looking for their cell phones 5 and selecting the cell phone or phones 5 of the rescuers 4 who are near the incident, meaning who are close to the monitoring device 2.

In a fifth step 14, the server 6 retrieves the fastest route between the nearest located defibrillator 3 and the monitoring device 2 of the victim 1, from the mapping system 9, and retrieves the fastest route or routes between the mobile phone or phones 5 of the rescuers 4 and the monitoring device 2 of the victim 1, from that same mapping system 9.

In a sixth step 15, the server 6 remotely triggers the alarm, for example an audible alarm and a flashing light, on the located defibrillator 3 in order to alert nearby rescuers 4, whether or not they are declared and identified in the database 7 and can then be notified via their cell phones 5 or are not declared and are simply passing near the located defibrillator 3. Simultaneously, the server 6 sends to the located defibrillator 3 the steps of the route to take to reach the victim 1 at the scene of the incident, advantageously a route from the located defibrillator 3 to the monitoring device 2 of this victim 1.

In a seventh step 16, the server 6 notifies the rescuers 4 declared as being in the vicinity, meaning those who are identified in the database 7 and who can be reached via their cell phones 5, for example via a mobile application on their smartphone if their phones 5 have it installed. To these mobile phones 5 are also sent the steps of the route to take to the located defibrillator 3 and to the victim 1 at the place of the incident, advantageously meaning a first route from their position to the located defibrillator 3 and a second route from the located defibrillator 3 to the monitoring device 2 of the victim 1. These first and second routes may advantageously be recalculated and updated in real time by the server 6, particularly in case of movement of the located defibrillator 3 as it is carried by a rescuer 4, who may or may not be declared.

In an eighth step 17, the rescuer or rescuers 4 notified via their cell phones 5 and/or one or more undeclared rescuers 4 who heard the alarm triggered at the located defibrillator 3, will collect the located defibrillator 3, being guided by the audible alarm and/or flashing light emitted by the located defibrillator 3 or by their mobile application for rescuers 4 having been notified by the server 6.

In a ninth step 18, the rescuer 4 who first reaches the located defibrillator 3 and carries it away is guided to the monitoring device 2 of the victim 1, meaning to the site of the incident, via the route displayed on a display of the located defibrillator 3 and/or via a voice interface of the located defibrillator 3, and/or via a mobile application on his or her cell phone 5 when the rescuer was notified by the server 6.

The located defibrillator 3 may also include a function for managing the return of the located defibrillator 3, said function calculating for example the route to the nearest return station.

Of course, the invention is not limited to the examples and the embodiment described and depicted, but is capable of numerous variants accessible to persons skilled in the art.

1. A method for managing a pool of emergency devices, comprising:
determining the location of a monitoring device carried by person experiencing a medical problem,
locating, in the pool of emergency devices, at least one emergency device near the located monitoring device,
indicating by an alarm at least the position of the located emergency device,
indicating a guide, at the located emergency device, to at least the position of the located monitoring device.

2. The method for managing a pool of emergency devices according to claim 1, also comprising a step of locating at least one telecommunications device carried by a rescuer who is near the located emergency device, and sending, to the located telecommunications device, at least the position of the located emergency device.

3. The method for managing a pool of emergency devices according to claim 2, wherein, in the sending step, a route to the located emergency device is sent to the located telecommunications device, this route preferably being recalculated in real time according to any movement of the located emergency device.

4. The method for managing a pool of emergency devices according to claim 2, wherein, in the sending step, at least the position of the located monitoring device is sent to the located telecommunications device, and a route to the located monitoring device is sent to the located telecommunications device.

5. The method for managing a pool of emergency devices according to claim 1, wherein, in the alarm step, a visual and/or audible alarm is triggered at the located emergency device, and in the guiding step at least a position of the located monitoring device is indicated at the located emergency device, preferably indicating a route to the located monitoring device.

6. The method for managing a pool of emergency devices according to claim 3, wherein said route or routes are the fastest in terms of travel time for the located emergency device and for the located telecommunications device, this travel time preferably being less than a predetermined duration, said predetermined duration even more preferably being less than 10 minutes or even less than 5 minutes.

7. The method for managing a pool of emergency devices according to claim 1, wherein, in the guiding step and in the sending step, other additional information may be indicated, including:
the position or positions of the one or more other located emergency devices, and the position or positions of the one or more other located telecommunications devices, and the medical qualification profile of the one or more other located telecommunications devices, and the identification of the monitoring device towards which the or each located emergency device is heading, and a confirmation of emergency response by one or more located telecommunications devices.

8. The method for managing a pool of emergency devices according to claim 1, wherein, in locating the telecommunications device, the located telecommunications device or devices are the telecommunications device or devices closest to the located emergency device, the located telecommunications device or devices preferably being determined from among a predetermined group of telecommunications devices carried by rescuers and registered.

9. The method for managing a pool of emergency devices according to claim 1, wherein the steps of the method take place in parallel with at least two located emergency devices and with at least two located telecommunications devices per located emergency device.

10. The method for managing a pool of emergency devices according to claim 1, wherein the movement of the located emergency device towards the located monitoring device and/or the arrival of the located emergency device at the located monitoring device, are indicated to the located telecommunications device and to other telecommunications devices carried by rescuers and alerted.

11. The method for managing a pool of emergency devices according to claim 1, wherein the step of locating a monitoring device is preceded by identifying this monitoring device by analyzing data sent by this monitoring device indicating a medical problem in the victim who is carrying said monitoring device.

12. The method for managing a pool of emergency devices according to claim 1, wherein locating a monitoring device is performed directly after transmission by the monitoring device of its geolocation, said monitoring device having itself performed a data analysis revealing a medical problem in the victim who is carrying said monitoring device.

13. The method for managing a pool of emergency devices according to claim 1, wherein the monitoring device sends data to a server which manages the pool of emergency devices within which the monitoring device is currently located, said server being able, through analysis of these data, to identify and locate said monitoring device.

14. The method for managing a pool of emergency devices according to claim 1, wherein two located telecommunications devices are placed in telecommunication with each other, the first located telecommunications device to reach the located monitoring device corresponding to a medical qualification profile that is lower than that of the second located telecommunications device which has not yet reached the located monitoring device.

15. The method for managing a pool of emergency devices according to claim 1, wherein the emergency device is a cardiac defibrillator.

16. A server for managing a pool of emergency devices, suitable for carrying out:
a step of determining the location of a monitoring device carried by a person experiencing a medical problem,
a step of locating, in the pool of emergency devices, an emergency device near the located monitoring device,
a step of sending, to the located emergency device, a command to trigger an alarm that indicates at least the position of the located emergency device,
a step of sending, to the located emergency device, a guiding instruction that indicates at least the position of the located monitoring device.

17. An emergency device intended to be placed in a pool of emergency devices, and adapted for carrying out, on a server for managing the pool of emergency devices, when said emergency device has been located as being close to a monitoring device carried by a victim experiencing a medical problem:
an alarm step, triggered by a command from the management server, that indicates at least the position of said located emergency device,
a guiding step that indicates at least the position of the monitoring device after receiving this position from the management server.
